(19) United States
(12) Patent Application Publication
(10) Pub. No.: US 2008/0227870 A1
(43) Pub. Date: Sep. 18, 2008

(54) Carcinogen Solvents in Reducing the Carcinogen Concentration of Cells

(76) Inventor: Kaizen Robert Matsumoto, London (GB)

Correspondence Address: GREENBERG TRAURIG, LLP, 1200 SEVENTEENTH STREET, SUITE 2400, DENVER, CO, 80202, US

(21) Appl. No.: 11/720,029

(22) PCT Filed: Dec. 1, 2005
(86) PCT No.: PCT/GB2005/004312

§ 371 Date: May 23, 2007

(30) Foreign Application Priority Data
Dec. 2, 2004 (GB) 0426489.1

(51) Int. Cl. A61K 31/02; A61P 35/00

(52) U.S. Cl. 514/759

(57) ABSTRACT

The present invention describes biologically compatible inert perfluorocarbon liquids capable of removing carcinogens from cells and thereby reducing the risk of cancerous cell transformation. This invention can be used to reduce the build-up of carcinogens, and thus be used as prophylactic medicaments in reducing the risk of chemically induced cancerous cell transformation and thus cancer. These liquids can be used as medicaments applied to any organ of the body; such an application would reduce the build-up of carcinogenic chemicals within cells of that organ and as such can be used to reduce the risk of a variety of cancers produced through chemical carcinogenesis. Such cancers include lung cancer, stomach cancer, rectal cancer, and certain types of skin cancer.

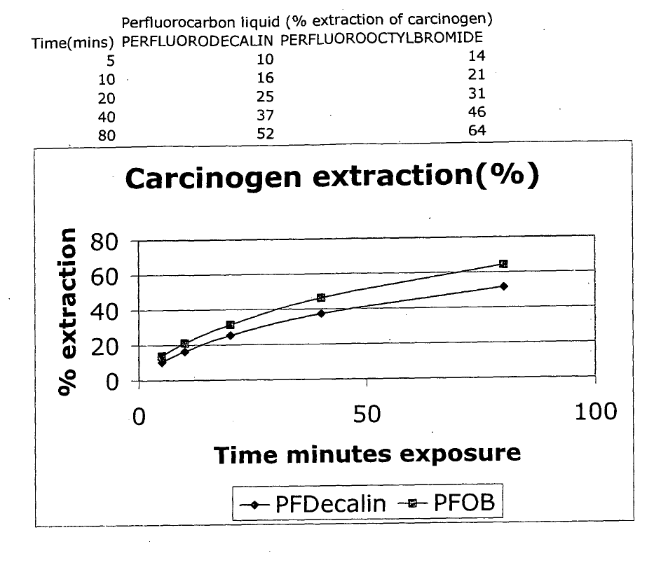

The invention relates to the use of perfluorocarbon liquids, as carcinogen solvents, in medicaments for the prophylactic removal of carcinogens from non-cancerous cells, for reducing the risk of cancerous cell transformation.

### BACKGROUND TO THE INVENTION

Carcinogens are known to accumulate within the lipophillic environment of the cellular membranes of cells. These accumulated carcinogens represent a carcinogenic reservoir that increases the risk of the cell becoming cancerous. The use of a carcinogen solvent can decrease the risk of cancerous transformation of the cell [1].

Increasing lipophillicity increases the carcinogenic potential of the carcinogen, as the carcinogen is present in the cell for a longer period and is not removed [2].

Carcinogens accumulate within the cellular membranes of cells due to a passive partitioning mechanism, the carcinogens are large organic molecules and therefore lipophillic, and the cellular membrane is phospholipid-based and therefore also lipophillic [3].

This principle of passive partitioning was used to explain the accumulation and localisation of lipophillic carcinogens within cellular membranes [4].

This passive partitioning mechanism is dependent on the relative volumes and lipophillicity of the external solvent and the cellular membranes [4].

The principle of passive partitioning also explained how lipophillic chemicals could be extracted from cellular membranes using lipoproteins in aqueous solution. Lipoproteins are proteins with lipid moieties; the lipid moieties create a lipophillic environment in which lipophillic carcinogens can dissolve, and thus the lipoproteins act as carcinogen solvents [5].

Exposing cellular membranes with intra-cellularly dissolved carcinogens to these lipoprotein solutions alters the relationship between the relative volumes and lipophillicity of the intra-cellular environment and the extra-cellular environment. If the extra-cellular environment, i.e. the lipoprotein solutions, is sufficiently lipophillic, the intra-cellularly dissolved carcinogens will passively partition out of the cells and into the extracellular lipoprotein solution, acting as a carcinogen solvent.

This extraction process was first and uniquely demonstrated in 1981 by Remsen and Shireman [5].

The experiment demonstrated that benzo(a)pyrene, a common environmental carcinogen, could be extracted with a variety of lipoprotein solutions at different concentration, it is also the sole experiment demonstrating the removal of internally dissolved carcinogens from cellular membranes using lipoprotein solutions as carcinogen solvents.

### DESCRIPTION OF THE INVENTION

This invention describes how the class of chemicals known as perfluorocarbon liquids can be used in a similar manner to lipoproteins, ie as carcinogen solvents, in the removal of carcinogens dissolved within the cells of the body. The removal of carcinogens from cells thereby reduces the risk of cancerous cell transformation.

No other extraction medium, ie carcinogen solvent, apart from aqueous-lipoprotein solutions and perfluorocarbon liquids is known to the art or has been reported in the known art as a potential extra-cellular carcinogen solvent for these lipophillic carcinogens.

Perfluorocarbon liquids are a class of chemicals defined as being any hydrocarbon where all hydrogen atoms have been substituted with an atom from the halide family such as fluorine, chlorine or bromine.

The presence of the large halide atoms makes all perfluorocarbon liquids inherently lipophillic and thus capable of dissolving out lipophillic carcinogens from cellular membranes, and being used in this invention, as carcinogen solvents.

Various perfluorocarbon are known within the literature, and are envisaged as appropriate for this invention, as all perfluorocarbons are inherently lipophillic.

The fundamental properties required for a perfluorocarbon to be used in this invention as a carcinogen solvent, is that the perfluorocarbon should be liquid and should be lipophillic.

### EXAMPLES

Two perfluorocarbon liquids, perfluorooctylbromide and perfluorodecalin, were tested for their ability to remove a model carcinogen, benzo(a)pyrene, from human fibroplasts. The chemical used, benzo(a)pyrene is an environmental carcinogen, produced in the incomplete combustion of organic materials. It is a lipophillic carcinogen, generally regarded as a good model for the behaviour of other such lipophillic carcinogens [5].

The cells were exposed to radiolabelled benzo(a)pyrene, after exposure the cells were washed with buffer. The cells were then exposed to the perfluorocarbon liquid. Aliquots of the perfluorocarbon liquid were removed at intervals and the radioactivity measured with a scintillation counter. After 120 minutes the cells were tested for membrane integrity with a vital dye, and the membrane and cells were found to be viable, for further reference on the biological effect of perfluorocarbons on phospholipid bilayers see Lack of effect of perfluorooctylbromide on phoshpolipid bilayers, [6]

Following this, the cells were dissolved in acid, and the radioactivity remaining in the cells was measured in a scintillation counter. These measurements were then used to calculate the relative carcinogen extraction in percentage terms against time of exposure to the perfluorocarbon liquid.

The perfluorocarbons were able to remove between 50% and 65% of the benzo(a)pyrene in an 80 minute exposure, see drawing 1/1, compared to lipoprotein solution which removed 43% and growth medium which removed 5%, data not shown.

Although the cell type used for this in vitro extraction was a human pulmonary cell type, it is envisaged that the invention will work on any cell type, from any organism, as all cellular membranes have similar lipophillic properties. The membranes of cells are necessarily lipophillic as they are made of phospholipids, where the lipid moieties form the intra-membrane space.

This invention thus describes that the perfluorocarbon liquids, perfluorooctylbromide, Perfluorodecalin and perfluorocarbon liquids in general can be used as biologically compatible carcinogen solvents in a formulation of a medicament for the removal of carcinogens from cells. The cells to be treated need to be exposed to the perfluorocarbon liquid medicament for an extended period of time, at least an hour, for the carcinogens to equilibrate between the intra-cellular environment and the extra-cellular carcinogen solvent, in the case of this invention a biologically inert perfluorocarbon liquid. Following equilibration of the carcinogen between the extra-cellular perfluorocarbon carcinogen solvent and the intra-cellular environment, the perfluorocarbon liquid can be removed, and thus effect the removal of carcinogens from cells.

Criteria for Choosing Suitable Perfluorocarbon Liquids for Use in this Invention.

### Lipophillicity

Perfluorocarbons deemed suitable for this invention would need to be lipophillic and generally insoluble in water. This is true for all perfluorocarbons, the substitution of hydrogen atoms by halide atoms creates the high lipophillicity of perfluorocarbon liquids.

### Boiling Point

The perfluorocarbon used would need to be liquid at normal body temperature, so that the cells can be exposed to the liquid form of the perfluorocarbon without inducing a temperature shock. Also, a perfluorocarbon gas cannot act as a carcinogen solvent.

### Biological Compatibility

The perfluorocarbon used would also need to be non-toxic to the cell; this is true for nearly all perfluorocarbon liquids, and true for the used examples, perfluorooctylbromide and perfluorodecalin.

Known perfluorocarbon liquids suitable for use in the human body and envisaged as proven/obvious candidates for use in this invention include;

Perfluorodecalin (F2 chemicals limited),
Perfluorooctylbromide (Exfluor research corporation),
FC-84 (Fluorinert™ 3M corporation),
FC-72 (Fluorinert™ 3M corporation),
FC-75 (Fluorinert™ 3M corporation),
RM-82 (Perflutel™ Miteni corporation),
RM-101 (Perflutel™ Miteni corporation).

This is list is not exhaustive and various other perfluorocarbons liquids have been tested for human medical use and can be deemed to be suitable for this invention, if they meet the lipophillicity criteria. Those skilled in the art of perfluorocarbon medicaments will appreciate that since all perfluorocarbon liquids are inherently lipophillic practically any existing perfluorocarbon liquid can be used as a carcinogen solvent in the removal of intra-cellularly dissolved carcinogens.

### Duration of Exposure

As the invention describes, the cells in need of carcinogen removal would need to be exposed for an extended period of time, an hour at least, preferably eighty to a hundred minutes, for the carcinogens to equilibrate between the intracellular environment and the perfluorocarbon medicament.

Following exposure of the cells by the perfluorocarbon medicament, the perfluorocarbon medicament should be removed, and thereby permanently remove the carcinogens from the cells. If the perfluorocarbon is left to evaporate the carcinogens will re-enter the cells, as the extra-cellular environment can longer dissolve them.

### Background on Perfluorocarbons

Perfluorocarbon liquids are known medicaments with various properties that make them suitable for use in the human body.

Perfluorocarbons are odourless, colourless and biologically inert. Their low boiling temperatures and high volatility mean that they do not accumulate in the body. Their extremely low toxicity means that any residual perfluorocarbon does not detrimentally affect the body, and their high chemical stability means that they do not spontaneously break down into toxic products. They are biologically inert, and do not interfere in biological pathways.

They have various other properties, which make them suitable for various therapies. Several uses for perfluorocarbon have been found since their first use as liquid ventilating mediums, these include uses such as liquid ventilation, blood substitutes, ultrasound-heating agent, pulmonary lavage agents, eye surgery, drug delivery agents, surfactant substitutes, radiograph contrast agents, and ultrasound contrast agents. Although the use of perfluorocarbon liquids as pulmonary lavage agents is part of the state-of-the-art, this inventions describes the use of perfluorocarbon based medicaments in the removal of carcinogens already dissolved into cells, not congestive materials which can not dissolve into the cells.

Furthermore, in the known use of perfluorocarbon liquids as lung lavage agents, the perfluorocarbon is only briefly exposed to the pulmonary tissue to remove congestive materials, which is insoluble in the perfluorocarbon liquid. The congestive material floats on top of the highly dense perfluorocarbon liquid, and does not dissolve into the perfluorocarbon liquid.

The use of perfluorocarbon lung lavage is for critical care use, where the patients' breathing is imminently threatened by a physical blockage in the airways. Perfluorocarbon lung lavage is performed in a very transient manner in which the perfluorocarbon liquid is used only to displace the airway blocking material; exposure of the cells to the perfluorocarbon liquid is measured in seconds and minutes. This invention does not relate to blockages of the airways, or as a critical care use of perfluorocarbon liquids. Furthermore the intra-cellularly dissolved carcinogens do not block the airways of the lung.

### PREFERRED EMBODIMENTS

### In Vivo Detoxification

In its preferred embodiment the invention would constitute a pure perfluorocarbon liquid medicament, either perfluorodecalin, perfluorooctylbromide or a lipophillic perfluorocarbon liquid chosen according to the stated criteria, used through exposing the cells in need of detoxification to the perfluorocarbon medicament for a period of at least one hour, preferably eighty to a hundred minutes, for maximum removal of carcinogens to occur. Following exposure the perfluorocarbon medicament should be removed from the organ as a liquid, and not left to evaporate.

The perfluorocarbon medicament can be applied directly to the cells in need of carcinogen removal. The methods and precautions needed for use of perfluorocarbon medicaments in the various organs of the body are known to those skilled in the art. Various organs of the body are known to have higher carcinogen concentrations, and these higher carcinogen concentrations can lead to cancerous cells and tumours. The perfluorocarbon used in the medicament could be any of several known perfluorocarbons with high lipophillicity. The perfluorocarbon would need to be otherwise biologically inert, and liquid at the temperature of treatment. In the case of human use, the perfluorocarbon would need to be liquid at body temperature.

Depending on the organ or tissue to be exposed, other properties of the perfluorocarbon medicament would need to be considered. For use in the lung tissue, or internal organs of the body, the perfluorocarbon liquid would need to be able to dissolve high amounts of gases. Selection criteria for a perfluorocarbon liquid capable of being used in the lung tissue or other internal organs of the body are obvious and known to the art.

For a review of potentially suitable perfluorocarbons for use in the body see Biro.p, Blais P: Perfluorocarbon blood substitutes, CRC critical reviews in oncology/haematology, Vol 6, No. 4, p 311-374, 1987.

### Diagnostic

As the perfluorocarbon liquid medicament is retrieved it can be analysed to quantify the presence of the removed carcinogens. Such quantification could be used as a diagnostic tool to quantify the life-long exposure to the various carcinogens, and the current risk associated with those doses.

### In-Vitro Detoxification

It is envisaged that the perfluorocarbon liquid medicament could be used in a laboratory setting to remove carcinogens from cells in culture.

Such a use could be useful in determining effective dose to the cells of the carcinogen, or determining metabolised proportion of the carcinogens, and other such uses. For in vitro use, the perfluorocarbon liquid need only be lipophillic and liquid at the temperature the experiment is conducted.

### REFERENCE

[1] High-density lipoproteins decrease both binding of a polynuclear aromatic hydrocarbon carcinogen to DNA and carcinogen-initiated cell transformation. Mutation research. 1983, November:111(3):p 429-439. D Busbee, W Benedict.
[2] Correlation of the octanol/water partition coefficient with clearance half-times of intratracheally instilled aromatic hydrocarbons in rats. Toxicology. 1985, September; 36(4) p 285-295. J. Bond, S. Baker, W. Bechtold.
[3] Mechanism and rate of permeation of cells by Polycyclic aromatic Hydrocarbons, The journal of biological chemistry, vol 262, No. 6, February, 1987, p 2514-2519. A. Plant, R. Knapp, L. Smith,
[4] Cellular uptake and intracellular localisation of benzo(a)pyrene by digital fluorescence imaging microscopy, The journal of cell biology, Volume 100, April 1985, p 1295-1308). A Plant, D Benson, L Smith
[5] Removal of benzo(a)pyrene from cells by various components of medium Cancer Letters, 1981 October:14(1) p 41-46 J Remsen, R Shireman:
[6] Lack of effect of perfluorooctylbromide on phospholipid bilayers. Biophysical journal (annual meeting abstracts) Ellena et al, 82(1) p 157
[7] Perfluorocarbon blood substitutes, CRC critical reviews in oncology/haematology, Vol 6, No. 4, p 311-374, 1987. Biro.p, Blais P:

1. A method for the removal of carcinogens from cells, the method comprising contacting said cells with a carcinogen solvent, wherein said carcinogen solvent is a perfluorocarbon liquid.

2. (canceled)

3. The method of claim 1, wherein the perfluorocarbon liquid is Perfluorooctylbromide.

4. The method of claim 1, wherein the perfluorocarbon liquid is Perfluorodecalin

5. The method of claim 1, wherein the perfluorocarbon liquid is FC-84.

6. The method of claim 1, wherein the perfluorocarbon liquid is FC-72.

7. The method of claim 1, wherein the perfluorocarbon liquid is RM-82.

8. The method of claim 1, wherein the perfluorocarbon liquid is FC-75.

9. The method of claim 1, wherein the perfluorocarbon liquid is RM-101.

10. The method of claim 1, wherein said cells are pulmonary cells.

11. The method of claim 1, wherein said cells are intestinal tract cells.

12. The method of claim 1, wherein said cells are skin cells.

13. The method of claim 1, wherein said cells are contained within a biopsy tissue.

14. The method of claim 1, wherein said cells are contacted with said perfluorocarbon liquid in vitro.

15. The method of claim 1, wherein said cells are contacted with said perfluorocarbon liquid in vivo.

16. The method of claim 15 wherein said cells are contacted with said perfluorocarbon liquid for at least one hour.

17. The method of claim 15 further comprising the step of removing said perfluorocarbon liquid from contact with said cells.

18. The method of claim 17 further comprising the step of analyzing said perfluorocarbon liquid for the presence of carcinogens following removal of said perfluorocarbon liquid.
